(19) United States
(12) Patent Application Publication
(10) Pub. No.: US 2010/0159178 A1
(43) Pub. Date: Jun. 24, 2010

(54) MOTOR VEHICLE FLUID LINE

(75) Inventors: Peter SEBOE, Frankfurt/Main (DE); Janos KERTESZ, Sulzbach (DE)

Correspondence Address: GREENBLUM & BERNSTEIN, P.L.C., 1950 ROLAND CLARKE PLACE, RESTON, VA, 20191, US

(73) Assignee: NORMA Germany GmbH, Maintal (DE)

(21) Appl. No.: 12/641,551

(22) Filed: Dec. 18, 2009

(30) Foreign Application Priority Data
Dec. 19, 2008 (DE) 10 2008 063 960.5

(51) Int. Cl. B32B 1/08; B29D 23/00; A61K 36/00

(52) U.S. Cl. 428/36.91; 525/55

(57) ABSTRACT

Motor vehicle fluid line for at least one of cooling water, oil or crankcase blow-by gases and method of forming the fluid line. The fluid line includes a pipe having a syndiotactic polystyrene in combination with at least one further plastic surrounding an interior.

### CROSS-REFERENCE TO RELATED APPLICATIONS

The present application claims priority under 35 U.S.C. §119 of German Patent Application No. 10 2008 063 960.5, filed on Dec. 19, 2008, the disclosure of which is expressly incorporated by reference herein in its entirety.

### BACKGROUND OF THE INVENTION

1. Field of the Invention

The invention relates to a motor vehicle fluid line for cooling water, oil or crankcase blow-by gases with a pipe that surrounds an interior.

2. Discussion of Background Information

The invention is described below based on a cooling water line, such as is used, for example, in order to cause cooling water to circulate in a circulation that comprises an engine and a heat exchanger. In order to ensure frost protection, the cooling water generally contains glycol. A material must therefore be used for the fluid line which is resistant to glycol and other constituents of the cooling water. When the engine is in operation, an increased temperature prevails in the cooling water circulation, which temperature is generally more than 100° C. In order to achieve this high temperature without the cooling water evaporating, a correspondingly high pressure is necessary. The cooling water line must therefore in addition be resistant to temperature and pressure to an adequate extent.

The same applies to a corresponding extent when the fluid line is used for other fluids, for example, oil, such as motor oil or transmission oil, or crankcase blow-by gases. A resistance to increased temperature and increased pressure is also necessary in this case.

As a rule, pipes or hoses of a rubber material are therefore used for a cooling water line. Although this rubber material has the necessary resistance to the increased temperature and the increased pressure as well as a resistance to the constituents of the cooling water, cooling water hoses of this type are relatively complex to produce and therefore expensive. Furthermore, the shaping is subject to certain restrictions.

### SUMMARY OF THE INVENTION

The present invention provides a replacement for a rubber line.

According to embodiments, a motor vehicle fluid line of the type mentioned at the outset includes a pipe having a syndiotactic polystyrene in combination with at least one further plastic.

With a line of this type, with a relatively free shaping and favorable manufacturing costs, a pipe is obtained that is sufficiently resistant to increased pressure and increased temperature. Because the syndiotactic polystyrene is combined with at least one further plastic, it is no longer necessary to rely on the syndiotactic polystyrene alone to meet all of the demands that are made on a corresponding fluid line. Instead, the pipe can be modified accordingly through the selection of suitable plastics. In particular, the impact strength can be improved and/or the resistance to heat aging can be increased. While polystyrene is generally rather unsuitable for a fluid line of this type, excellent suitability surprisingly results when it is present in crystalline form as a syndiotactic polystyrene.

Preferably, the syndiotactic polystyrene and the further plastic form a layer-like structure. With a layering of the materials used, it is relatively simple to combine the syndiotactic polystyrene with other plastics. In this manner, the further plastic or the further plastics can be used precisely where they are necessary or useful.

Moreover, the pipe may preferably be formed as a co-extruded element of syndiotactic polystyrene and the further plastic. The co-extrusion makes it possible to combine the syndiotactic polystyrene and the further plastic or plastics with one another in a simple manner.

The syndiotactic polystyrene and the further plastic can preferably have an adhesion promoter. The adhesion promoter then ensures that the syndiotactic polystyrene and the further plastic undergo a permanent mechanical connection to one another, which also ensures an adequate mechanical stability over a longer period of time.

In an alternative embodiment, it can be provided that the syndiotactic polystyrene and the further plastic adhere directly to one another. In this case, it can be useful and necessary to modify the syndiotactic polystyrene such that a direct adhesion is obtained.

Preferably, the further plastic is a thermoplastic elastomer, in particular on a polyolefin or polyester basis. A sufficient number of materials are thus available which can be combined with the syndiotactic polystyrene. Thermoplastic elastomers can easily be brought into a desired form. In this manner, they ensure that the fluid line has a good impact strength.

Preferably, the further plastic is selected from a group that comprises PPE (polyphenylene ether), TPE (thermoplastic elastomer), PA (polyamide), EVA (ethylenevinyl acetate), PPS (polyphenylene sulfide), PP (polypropylene), PBT (polybutylene terephthalate) and PET (polyethylene terephthalate). PPE, TPE, PA, EVA, PPS, PP, PBT and PET are materials that can be shaped jointly with the syndiotactic polystyrene with reasonable expenditure. Certain mechanical properties can be adjusted in a targeted manner by selection of one or more materials from the above group to form the line. By way of example, the impact strength can be improved with TPE and EVA, while the other cited plastics produce an improvement in the resistance to heat aging, the tensile strength and/or the modulus of elasticity.

Preferably, the syndiotactic polystyrene is arranged on an inner wall delimiting the interior. In this case, the syndiotactic polystyrene is in direct contact with the fluid flowing through, for example, cooling water, oil or crankcase blow-by gases. An embodiment of this type is always possible when the syndiotactic polystyrene is compatible with the fluid. An inner wall formed with syndiotactic polystyrene is advantageous in particular in the case of a water/glycol mixture. A high dimensional stability is then produced.

In an alternative embodiment it can be provided that the syndiotactic polystyrene is arranged inside a sandwich structure of several layers. In this case, the syndiotactic polystyrene is covered by other plastics on the inside of the pipe and optionally also on the outside, so that it needs to be ensured only that the other plastic on the inside is compatible with the fluid flowing through. The syndiotactic polystyrene can then take on mechanical functions and make the fluid line resistant to temperature and pressure.

It is preferred hereby for the syndiotactic polystyrene to form the second layer from the inside. In this case, the syndiotactic polystyrene can best develop its effect. Only the inner layer, which is in contact with the fluid flowing through, needs to be resistant to the fluid flowing through. The main function of the line, of guiding the fluid even at higher temperatures and higher pressure, is then carried out by the layer formed of syndiotactic polystyrene.

It is hereby preferable that the inner layer includes PPS, TPE and/or PPE. In this regard, PPS, which has a reduced mechanical strength and is also generally somewhat more expensive in comparison to the syndiotactic polystyrene, is resistant to more fluids than syndiotactic polystyrene. PP/EPDM (polypropylene/ethylene propylene diene terpolymers), SBS (styrene/butadiene/styrene triblock copolymers) or SEBS (styrene/ethylene/butadiene/styrene), for example, are possible as a TPE.

Preferably, the syndiotactic polystyrene is reinforced with fibers. Glass fibers or carbon fibers, for example, are possible as the reinforcing fibers. These fibers are further used to increase the mechanical strength of the syndiotactic polystyrene.

It is also advantageous if at least one plastic is an acid-modified functionalized plastic, which can be, e.g., the syndiotactic polystyrene. This plastic then bonds better to the adjacent plastic. It becomes more polar.

Embodiments of the invention are directed to a fluid line that includes a pipe formed of syndiotactic polystyrene in combination with at least one further plastic surrounding an interior.

According to embodiments, the pipe may be structured and arranged as a motor vehicle fluid line for one of cooling water, oil or crankcase blow-by gases.

In accordance with further embodiments, the syndiotactic polystyrene and the further plastic can be combined to form a layer-like structure.

According to aspect of the invention, the pipe may be formed as a co-extruded element of the syndiotactic polystyrene and the further plastic.

According to further aspects of the invention, the pipe can be further formed with an adhesion promoter.

In accordance with still further embodiments, the syndiotactic polystyrene and the further plastic can be directly adhered to one another.

According to further aspects, the further plastic can include a thermoplastic elastomer. The thermoplastic elastomer comprises a polyolefin or polyester basis.

Moreover, the further plastic may include at least one of PPE, TPE, PA, EVA, PPS, PP, PBT and PET.

According to still further aspects, the syndiotactic polystyrene can form an inner wall of the pipe delimiting the interior.

In accordance with further embodiments, the syndiotactic polystyrene can be arranged inside a sandwich structure of several layers. The syndiotactic polystyrene can form a second layer from an innermost layer delimiting an interior. Further, the innermost layer may include PPS, TPE and/or PPE.

According to still further embodiments, the syndiotactic polystyrene may be reinforced with fibers.

Further, the at least one plastic can be an acid-modified functionalized plastic.

Embodiments of the invention are directed to a method for forming a fluid line. The method includes forming a cylinder from a combination of syndiotactic polystyrene and at least one further plastic.

According to embodiments of the invention, the fluid line can be a motor vehicle fluid line for one of cooling water, oil or crankcase blow-by gases

According to further embodiments, the at least one further plastic can include at least one of PPE, TPE, PA, EVA, PPS, PP, PBT and PET.

Moreover, the syndiotactic polystyrene and the at least one further plastic can be arranged in layers, and the syndiotactic can form an inner wall of the cylinder.

In accordance with still yet other embodiments of the invention, the syndiotactic polystyrene is forms a layer sandwiched between a first layer comprising the at least one further plastic and a second plastic layer

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 illustrates a diagrammatic representation of a fluid line;

FIG. 2 illustrates a sectional view along section II-II depicted in FIG. 1; and

FIG. 3 illustrates a modification of the sectional view depicted in FIG. 2.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

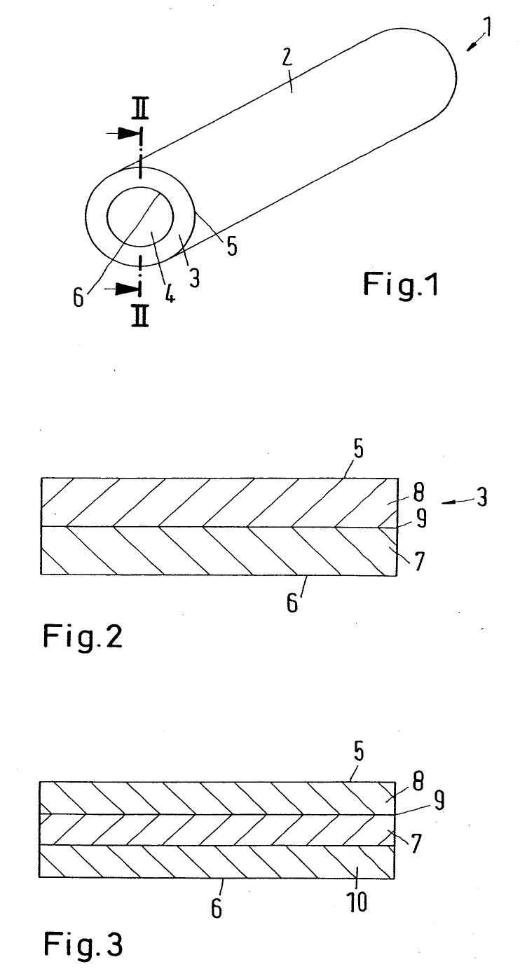

FIG. 1 shows a motor vehicle fluid line 1 for cooling water, oil or crankcase blow-by gases with a pipe 2, which has a wall 3 surrounding an interior 4. Pipe 2 has an outside surface 5 and an inside surface 6.

The wall 3 of pipe 2 is embodied or formed in a multilayered manner, as is shown by FIGS. 2 and 3.

According to a first exemplary embodiment illustrated in FIG. 2, inside surface 6 of wall 3 has a layer 7 of syndiotactic polystyrene (SPS). Layer 7 is covered on its outside, i.e., the surface opposite the surface 6, with a second layer 8 formed of a further plastic. An adhesion promoter 9 can be arranged between two layers 7 and 8. However, it is also contemplated that the syndiotactic polystyrene can be modified in such a manner that the further plastic of second layer 8 adheres directly or indirectly to layer 7. Thus, for example, the syndiotactic polystyrene can functionalize in an acid-modified manner.

The further plastic is preferably a thermoplastic elastomer, in particular on a polyolefin or polyester basis. Preferably, the further plastic of the second layer 8 is selected from a group that includes PPE (polyphenylene ether), TPE (thermoplastic elastomer), PA (polyamide), EVA (ethylenevinyl acetate), PPS (polyphenylene sulfide), PP (polypropylene), PBT (polybutylene terephthalate) and PET (polyethylene terephthalate).

Two layers 7 and 8 can be formed together by of co-extrusion. The syndiotactic polystyrene of first layer 7 is thereby resistant to increased temperature and increased pressure within a broad range. Therefore, the syndiotactic polystyrene of first layer 7 contributes to a large extent to the mechanical stability of line 1 in operation. The further plastic of second layer 8 can then be used for other purposes, for example, in order to ensure a good impact strength or attachability of line 1. The other plastic can be alternatively or additionally functionalized to the syndiotactic polystyrene, for example, acid-modified, in order to achieve a better adhesion.

FIG. 3 shows a modified embodiment, in which the same elements are provided with the same reference numbers. Layer 7 of the syndiotactic polystyrene is now arranged in a sandwich structure of several layers and forms the second layer of wall 3 seen from inside surface 6. A third layer is arranged as an innermost layer to form inside surface 6. This third layer can likewise be made from one of the plastics in the group referenced above. However, this layer is preferably made from PPS (polyphenylene sulfide).

First layer 7 of syndiotactic polystyrene can be reinforced with fibers, for example, with glass fibers or carbon fibers. The other two layers 8 and 10 can naturally also be reinforced with fibers.

Both embodiments are embodied or formed so that they endure at a temperature of 135° C. over a period of 700 hours in a water/glycol mixture and then withstand a low-temperature impact strength test according to Charpy (impact strength).

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. A fluid line comprising:
a pipe formed of syndiotactic polystyrene in combination with at least one further plastic surrounding an interior.

2. The fluid line in accordance with claim 1, wherein the pipe is structured and arranged as a motor vehicle fluid line for one of cooling water, oil or crankcase blow-by gases.

3. The fluid line in accordance with claim 1, wherein the syndiotactic polystyrene and the further plastic are combined to form a layer-like structure.

4. The fluid line in accordance with claim 1, wherein the pipe is formed as a co-extruded element of the syndiotactic polystyrene and the further plastic.

5. The fluid line in accordance with claim 1, wherein the pipe is further formed with an adhesion promoter.

6. The fluid line in accordance with claim 1, wherein the syndiotactic polystyrene and the further plastic directly adhere to one another.

7. The fluid line in accordance with claim 1, wherein the further plastic comprises a thermoplastic elastomer.

8. The fluid line in accordance with claim 1, wherein the thermoplastic elastomer comprises a polyolefin or polyester basis.

9. The fluid line in accordance with claim 1, wherein the further plastic comprises at least one of PPE, TPE, PA, EVA, PPS, PP, PBT and PET.

10. The fluid line in accordance with claim 1, wherein the syndiotactic polystyrene forms an inner wall of the pipe delimiting the interior.

11. The fluid line in accordance with claim 1, wherein the syndiotactic polystyrene is arranged inside a sandwich structure of several layers.

12. The fluid line in accordance with claim 11, wherein the syndiotactic polystyrene forms a second layer from an innermost layer delimiting an interior.

13. The fluid line in accordance with claim 12, wherein the innermost layer comprises PPS, TPE and/or PPE.

14. The fluid line in accordance with claim 1, wherein the syndiotactic polystyrene is reinforced with fibers.

15. The fluid line in accordance with claim 1, wherein the at least one further plastic is an acid-modified functionalized plastic.

16. A method for forming a fluid line, comprising:
forming a cylinder from a combination of syndiotactic polystyrene and at least one further plastic.

17. The method in accordance with claim 16, wherein the fluid line is a motor vehicle fluid line for one of cooling water, oil or crankcase blow-by gases.

18. The method in accordance with claim 16, wherein the at least one further plastic comprises at least one of PPE, TPE, PA, EVA, PPS, PP, PBT and PET.

19. The method in accordance with claim 16, wherein the syndiotactic polystyrene and the at least one further plastic are arranged in layers, and the syndiotactic forms an inner wall of the cylinder.

20. The fluid line in accordance with claim 16, wherein the syndiotactic polystyrene is forms a layer sandwiched between a first layer comprising the at least one further plastic and a second plastic layer.
